(19) United States
(12) Patent Application Publication
(10) Pub. No.: US 2026/0341532 A1
(43) Pub. Date: Oct. 29, 2026

(54) METHODS AND SYSTEMS FOR IMPROVED ITERATIVE DATA EXTRACTION FROM ELECTRONIC HEALTH RECORDS

(71) Applicant: KONINKLIJKE PHILIPS N.V., EINDHOVEN (NL)

(72) Inventor: YALE CHANG, BEDFORD, MA (US)

(21) Appl. No.: 19/107,108

(22) PCT Filed: Sep. 8, 2023
(86) PCT No.: PCT/EP2023/074702

§ 371 Date: Feb. 27, 2025

Related U.S. Application Data
(60) Provisional application No. 63/405,536, filed on Sep. 12, 2022.

(51) Int. Cl. G16H 50/70; G16H 10/60; G16H 50/20

(52) U.S. Cl. CPC G16H 50/70; G16H 10/60; G16H 50/20

(57) ABSTRACT

A method for generating a modified patient cohort, comprising: receiving a plurality of patient records for a plurality of patients; retrieving, from a clinical feature database, a predetermined plurality of features for a clinical diagnosis, condition, or disease; extracting, using the predetermined plurality of features, clinical features from the patient records to generate a clinical feature dataset; generating a feature value table comprising the extracted clinical feature dataset; identifying, based on one of the plurality of clinical diagnoses, conditions, or diseases, a first patient cohort from the feature value table; providing the first patient cohort to the user; receiving a modification request from the user to modify the first patient cohort; modifying, without additional extraction of clinical features, the first patient cohort to generate a modified patient cohort; and providing the modified patient cohort to the user.

### FIELD OF THE DISCLOSURE

The present disclosure is directed generally to methods and systems for iterative data extraction and patient cohort generation from electronic health records.

### BACKGROUND

Electronic health record databases comprise medical records and information for a very large plurality of patients. Mining these databases for information about illnesses, conditions, diseases, treatments, and other aspects of health and well-being has become a very common practice in medical research and treatment. For example, identifying and extracting a patient cohort of interest from the plurality of patients in an electronic health record database is often a first step toward developing a disease risk prediction model, a treatment pathway, and many other research models and analyses.

To identify a patient cohort of interest from the plurality of patients in an electronic health record database, investigators need to first extract the patient cohort, which consists of input variable measurements and disease labels, from the data source. There will typically be: (i) a data source; (ii) labeling criteria; and (iii) a list of input variables as input for identification and extraction.

As just one example, in order to build a sepsis prediction model, an investigator can start from a MIMIC-III database (i.e., the data source), apply sepsis III labelling criteria of sepsis (i.e., the labelling criteria), and then extract a list of input variables that are potentially predictive of sepsis, such as white blood cell count, lactate, etc. (i.e., list of input variables). This prior art process is illustrated in flowchart 10 in FIG. 1.

However, this data extraction is often a time-consuming and iterative process. For example, during an iterative process, any of the following might be modified: 1) patient dimension; 2) feature dimension; and 3) time dimension. For example, both patients and features can be added, removed, or updated. The time window can be updated for both input data extraction and data imputation. Since running the data extraction process shown in FIG. 1 is so time-consuming, it is extremely inefficient to rerun the entire identification and extraction process given even a minor modification in any of patient/feature/time dimensions.

### SUMMARY OF THE DISCLOSURE

Accordingly, there is a continued need for methods and systems for faster and more efficient iterative data extraction and patient cohort generation from electronic health records. Various embodiments and implementations herein are directed to a patient cohort creation system configured to generate a modified patient cohort from a database comprising a plurality of patient records for a plurality of patients. The patient cohort creation system receives a plurality of patient records for a plurality of patients from a database comprising. The system also retrieves, from a clinical feature database, a predetermined plurality of features for a clinical diagnosis, condition, or disease, wherein the clinical feature database comprises a predetermined plurality of features for each of a plurality of clinical diagnoses, conditions, or diseases. The method further includes extracting, using the predetermined plurality of features for the clinical diagnosis, condition, or disease, clinical features from at least some of the plurality of patient records for the plurality of patients to generate a clinical feature dataset for a first time period; generating a feature table in memory comprising the extracted clinical feature dataset; identifying, in response to a user request for a patient cohort based on one of the plurality of clinical diagnoses, conditions, or diseases, a first patient cohort from the feature table for a predetermined time period, wherein the predetermined time period is some or all of the first time period, and wherein the first patient cohort is a subset of the plurality of patients in the feature table; providing the first patient cohort to the user; receiving a modification request from the user to modify the first patient cohort, wherein the modification comprises a modification of one or more of: (i) patients in the first patient cohort; (ii) the predetermined time period; and (iii) the predetermined plurality of features for the clinical diagnosis, condition, or disease; modifying, based on the received modification request and without additional extraction of clinical features, the first patient cohort to generate a modified patient cohort; and providing the first patient cohort to the user.

Generally, in one aspect, a method for generating a modified patient cohort from a database comprising a plurality of patient records for a plurality of patient is provided. The method includes: receiving, from the database, the plurality of patient records for the plurality of patients; retrieving, from a clinical feature database, a predetermined plurality of features for a clinical diagnosis, condition, or disease, wherein the clinical feature database comprises a predetermined plurality of features for each of a plurality of clinical diagnoses, conditions, or diseases; extracting, using the predetermined plurality of features for the clinical diagnosis, condition, or disease, clinical features from at least some of the plurality of patient records for the plurality of patients to generate a clinical feature dataset for a first time period; generating a feature value table in memory comprising the extracted clinical feature dataset; identifying, in response to a user request for a patient cohort based on one of the plurality of clinical diagnoses, conditions, or diseases, a first patient cohort from the feature value table for a predetermined time period, wherein the predetermined time period is some or all of the first time period, and wherein the first patient cohort is a subset of the plurality of patients in the feature value table; providing, via a user interface, the first patient cohort to the user; receiving a modification request from the user to modify the first patient cohort, wherein the modification comprises a modification of one or more of: (i) patients in the first patient cohort; (ii) the predetermined time period; and (iii) the predetermined plurality of features for the clinical diagnosis, condition, or disease; modifying, based on the received modification request and without additional extraction of clinical features, the first patient cohort to generate a modified patient cohort; and providing, via the user interface, the modified patient cohort to the user.

According to an embodiment, the feature value table comprises, for each of a plurality of patients, a patient identification and a feature value for some or all of the predetermined plurality of features for the clinical diagnosis, condition, or disease.

According to an embodiment, the first patient cohort and the modified patient cohort comprises a list of patients each identified by a patient ID, and, for each patient, a feature value for each of the predetermined plurality of features.

According to an embodiment, the modified patient cohort comprises more patients compared to the first patient cohort.

According to an embodiment, the modified patient cohort comprises one or more additional features compared to the first patient cohort.

According to an embodiment, the modified patient cohort comprises a longer or shorter time period compared to the first patient cohort.

According to an embodiment, the modification request received from the user comprises an identification of a feature not originally listed in the predetermined plurality of features for the clinical diagnosis, condition, or disease.

According to an embodiment, the predetermined time period is a time period before onset of a clinical condition.

According to another aspect is a system for generating a modified patient cohort. The system includes: a database comprising a plurality of patient records for a plurality of patients; a clinical feature database comprising a predetermined plurality of features for each of a plurality of clinical diagnoses, conditions, or diseases; a user interface; and a processor, the processor configured to: (i) retrieve, from a clinical feature database, a predetermined plurality of features for a clinical diagnosis, condition, or disease; (ii) extract, using the predetermined plurality of features for the clinical diagnosis, condition, or disease, clinical features from at least some of the plurality of patient records for the plurality of patients to generate a clinical feature dataset for a first time period; (iii) generate a feature value table in memory comprising the extracted clinical feature dataset; (iv) identify, in response to a user request for a patient cohort based on one of the plurality of clinical diagnoses, conditions, or diseases, a first patient cohort from the feature value table for a predetermined time period, wherein the predetermined time period is some or all of the first time period, and wherein the first patient cohort is a subset of the plurality of patients in the feature value table; (v) provide, via the user interface, the first patient cohort to the user; (vi) receive a modification request from the user to modify the first patient cohort, wherein the modification comprises a modification of one or more of: patients in the first patient cohort; the predetermined time period; and the predetermined plurality of features for the clinical diagnosis, condition, or disease; (vii) modify, based on the received modification request and without additional extraction of clinical features, the first patient cohort to generate a modified patient cohort; and (viii) provide, via the user interface, the modified patient cohort to the user.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. The figures showing features and ways of implementing various embodiments and are not to be construed as being limiting to other possible embodiments falling within the scope of the attached claims. Also, the drawings are not necessarily to scale, emphasis instead generally being placed upon illustrating the principles of the various embodiments.

FIG. 1 is a flowchart of a prior art method for generating a patient cohort from a database.

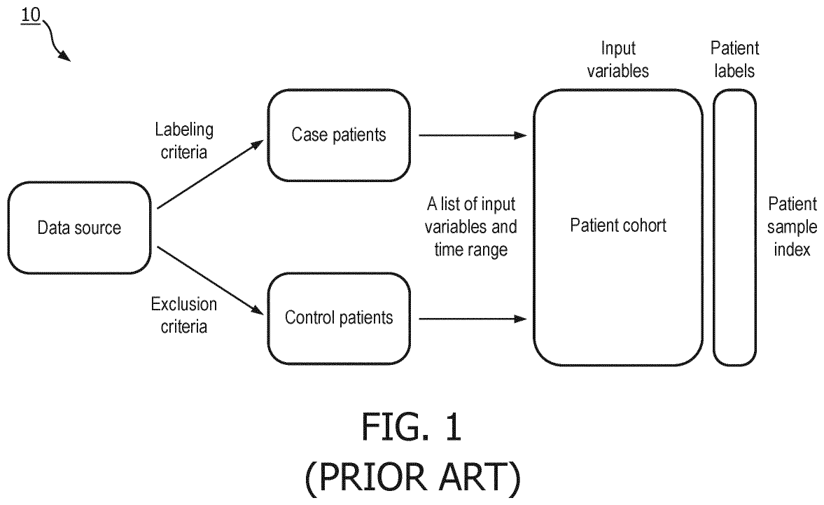

FIG. 2 is a flowchart of a method for generating a patient cohort from a database, in accordance with an embodiment.

FIG. 3 is a schematic representation of a patient cohort creation system, in accordance with an embodiment.

FIG. 4 is a schematic representation illustrating complexities induced by modifications of a patient cohort in three dimensions, in accordance with an embodiment.

FIG. 5 is a flowchart of a method for generating a patient cohort from a database, in accordance with an embodiment.

### DETAILED DESCRIPTION OF EMBODIMENTS

The present disclosure describes various embodiments of a system and method configured to identify and eliminate one or more clinical concerns from a clinical risk prediction model. More generally, Applicant has recognized and appreciated that it would be beneficial to provide an improved clinical risk prediction model or tool that is more generalizable to risk prediction for in-house deployment. A clinical risk prediction system receives or obtains a training dataset configured to train a clinical risk prediction model, the training dataset comprising The present disclosure describes various embodiments of a system and method configured to facilitate the rapid and efficient generation and modification of a patient cohort. More generally, Applicant has recognized and appreciated that it would be beneficial to provide an improved patient cohort creation system that creates a patient cohort from a database comprising a plurality of patient records for a plurality of patients. The patient cohort creation system receives a plurality of patient records for a plurality of patients from a database comprising. The system also retrieves, from a clinical feature database, a predetermined plurality of features for a clinical diagnosis, condition, or disease, wherein the clinical feature database comprises a predetermined plurality of features for each of a plurality of clinical diagnoses, conditions, or diseases. The method further includes extracting, using the predetermined plurality of features for the clinical diagnosis, condition, or disease, clinical features from at least some of the plurality of patient records for the plurality of patients to generate a clinical feature dataset for a first time period; generating a feature table in memory comprising the extracted clinical feature dataset; identifying, in response to a user request for a patient cohort based on one of the plurality of clinical diagnoses, conditions, or diseases, a first patient cohort from the feature table for a predetermined time period, wherein the predetermined time period is some or all of the first time period, and wherein the first patient cohort is a subset of the plurality of patients in the feature table; providing the first patient cohort to the user; receiving a modification request from the user to modify the first patient cohort, wherein the modification comprises a modification of one or more of: (i) patients in the first patient cohort; (ii) the predetermined time period; and (iii) the predetermined plurality of features for the clinical diagnosis, condition, or disease; modifying, based on the received modification request and without additional extraction of clinical features, the first patient cohort to generate a modified patient cohort; and providing the first patient cohort to the user.

The embodiments and implementations disclosed or otherwise envisioned herein can be utilized with any research or patient care system, including but not limited to clinical decision support tools, among other systems. For example, one application of the embodiments and implementations herein is to improve systems such as, e.g., the Philips® IntelliSpace® products (manufactured by Koninklijke Philips, N.V.), among many other products. However, the disclosure is not limited to these devices or systems, and thus disclosure and embodiments disclosed herein can encompass any device or system for which a patient cohort may be desirable or applicable.

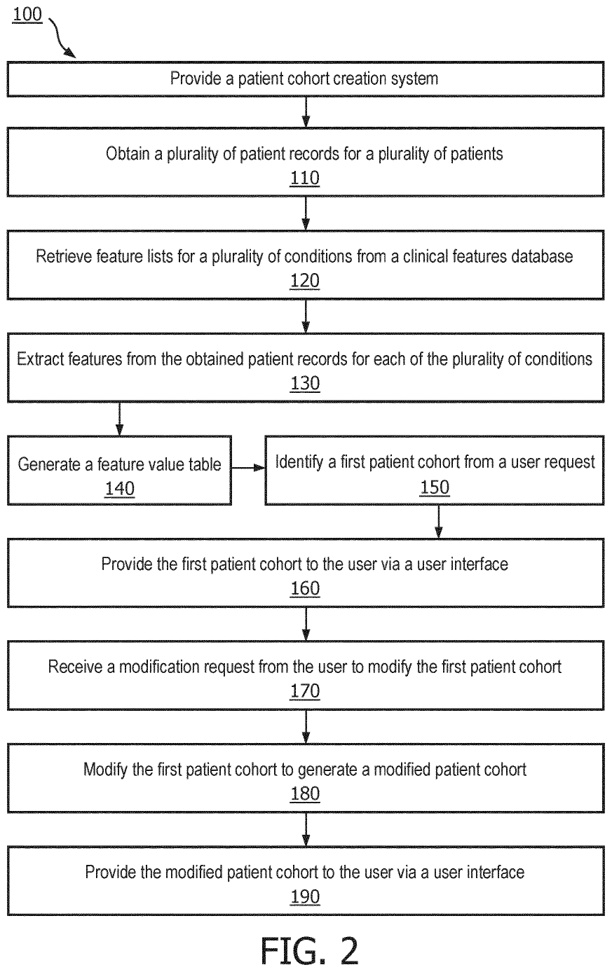

Referring to FIG. 2, in one embodiment, is a flowchart of a method 100 for generating a modified patient cohort from a database comprising a plurality of patient records for a plurality of patients, using a patient cohort creation system. The methods described in connection with the figures are provided as examples only, and shall be understood not to limit the scope of the disclosure. The patient cohort creation system can be any of the systems described or otherwise envisioned herein. The patient cohort creation system can be a single system or multiple different systems.

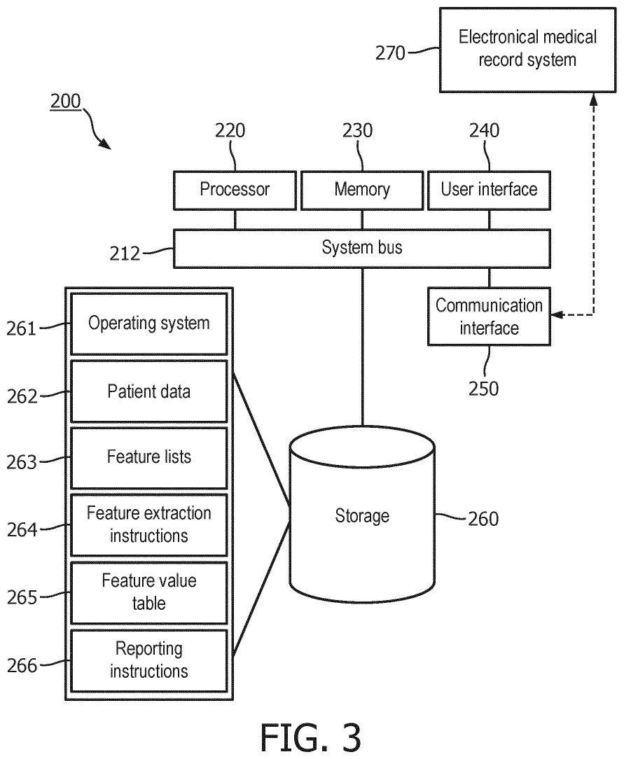

As an initial step of the method, a patient cohort creation system is provided. Referring to an embodiment of a patient cohort creation system 200 as depicted in FIG. 3, for example, the system comprises one or more of a processor 220, memory 230, user interface 240, communications interface 250, and storage 260, interconnected via one or more system buses 212. It will be understood that FIG. 3 constitutes, in some respects, an abstraction and that the actual organization of the components of the system 200 may be different and more complex than illustrated. Additionally, patient cohort creation system 200 can be any of the systems described or otherwise envisioned herein. Other elements and components of the patient cohort creation system 200 are disclosed and/or envisioned elsewhere herein.

At step 110 of the method, the patient cohort creation system 200 receives, retrieves, or otherwise obtains a plurality of patient records for a plurality of patients from a database 270. The database can be any database, including but not limited to an electronic health record database. For example, the database can be any database comprising health records for a multitude of patients or subjects. The database 270 may be a local or remote database and is in direct and/or indirect communication with the patient cohort creation system 200. The plurality of patient records may be stored in and/or received from one or more databases. Any of these databases may be a local and/or remote database. For example, the patient cohort creation system may comprise a database 270 of patient records.

The patient records can comprise any data about a patient. For example, the patient records may comprise demographic information, diagnostic information, clinical measurements, treatment information, and/or any other information, including over a period of time for each of the plurality of patients. According to an embodiment, the patient records comprises clinical data that measures patient physiology-such as physiological measurements of vital signs, lab results, imaging results, and other data- and event logs-such as timestamps of orders of labs, imaging, medication, consultation, and other data-that can reflect the procedures the patient received in a care setting. The patient records can comprise any other information.

According to an embodiment, the patient cohort creation system may comprise a data pre-processor or similar component or algorithm configured to process the received training data. For example, the data pre-processor analyzes the patient records to remove noise, bias, errors, and other potential issues. The data pre-processor may also analyze the input data to remove low-quality data. Many other forms of data pre-processing or data point identification and/or extraction are possible.

Once patient cohort creation system 200 receives, retrieves, or otherwise obtains the plurality of patient records for the plurality of patients from the database 270, the patient records can be utilized immediately, or may be stored in local and/or remote memory for future use.

Once the patient cohort creation system has a plurality of patient records, it can generate a database of extracted features for each of a plurality of different conditions, illnesses, diagnoses, treatments, and so on. To generate this database of extracted features, the patient cohort creation system must identify or otherwise comprise or receive or obtain a list of features that should be extracted for each of the different conditions, illnesses, diagnoses, and treatments. For example, for condition X, it may be well-known or anticipated that extracted features X1, X2, and X3 are often relevant to condition X. Similarly, for condition Y, it may be well-known or anticipated that extracted features Y1 and Y2 are often relevant to condition Y. And finally, for condition Z, it may be well-known or anticipated that extracted features X1, X3, and Y2 are often relevant to condition Z.

According to an embodiment, the plurality of different conditions, illnesses, diagnoses, treatments, and so on can comprise anything relevant to patient care, diagnosis, or treatment. For example, a condition can be sepsis, shock such as cardiogenic shock, septic shock, hypovolemic shock, acute kidney injury, acute respiratory distress syndrome, and hemodynamic instability, among a wide variety of other conditions.

According to an embodiment, a feature can be anything found within or otherwise obtainable from or determinable from a medical record or other patient record. A few non-limiting examples of features include demographics (age, sex, weight, height, etc.), diagnosis (diabetes, heart failure, lung injury, kidney injury, broken wrist, appendicitis, etc.), treatment (antibiotic course, appendectomy, dialysis, etc.), and much more. A feature can be almost anything that: (i) can be extracted from or otherwise identified within a patient record; and (ii) can be utilized by a researcher, clinician, or machine-learning algorithm to examine or study or learn about patient care, diagnosis, or treatment, among other things.

In addition, the identified, received, or obtained list of features that will be extracted from the plurality of patient records can be a comprehensive list of features that comprise many or all features that may be known to be relevant to any one of a very wide and varied list of conditions, illnesses, diagnoses, treatments, and so on. This can be especially important for research and discovery, as it is often that case that some features may be relevant to a condition, illness, diagnosis, or treatment but that relevance may not yet be discovered or previously correlated. Accordingly, a wide array of extracted features should be available to a clinician in order to enable research and discovery.

Further, in addition to identifying patients and features relevant to each of the very wide and varied list of conditions, illnesses, diagnoses, treatments, and so on (i.e., a patient cohort), researchers or clinicians must also identify a comparison or control group. This comparison or control group should comprise patients that are phenotypically similar to those in the patient cohort, but might lack the condition, illness, diagnosis, treatment, and so on. Ideally, the comparison or control group should be similar to size or number to the patient cohort. Accordingly, having features extracted for a plurality of patients including both the ultimate patient cohort and the comparison or control group is important for the quick and efficient performance of the patient cohort creation system.

Accordingly, at step 120 of the method, the patient cohort creation system retrieves a list of features for each of a plurality of different clinical diagnoses, conditions, or diseases, from a clinical features database. The clinical features database comprises a predetermined plurality of features for each of the plurality of clinical diagnoses, conditions, and diseases. The clinical features database can be a local or remote database and can be in direct and/or indirect communication with the patient cohort creation system 200. The feature lists for the plurality of different clinical diagnoses, conditions, or diseases may be stored in and/or received from one or more databases. Any of these databases may be a local and/or remote database. For example, the patient cohort creation system may comprise a clinical features database.

For example, the clinical features database comprises a list of features Lx relevant to condition X, where the list of features Lx comprises a plurality of features that are known to be relevant to, or predicted to be relevant to, or otherwise associated with, condition X. List of features Lx may be, for example, ten different features for condition X (age, sex, antibiotic regimen, etc.). Similarly, the clinical features database comprises a list of features Lz relevant to condition Z, where the list of features Lz comprises a plurality of features that are known to be relevant to, or predicted to be relevant to, or otherwise associated with, condition Z. And so on for a wide variety of different diagnoses, conditions, illnesses, or diseases, and so on.

The clinical features database may also comprise a list of features Gn (“Generic”) which are not necessarily associated with a particular condition, but may be anticipated to be useful for research and discovery. The list of generic features can be any feature that can be extracted from patient records. Notably, some or all of the lists such as Lx, Lz and Gn can be partially or completely overlapping.

Once patient cohort creation system 200 receives, retrieves, or otherwise obtains the lists of predetermined plurality of features for the different clinical diagnoses, conditions, or diseases from the clinical feature database, the lists can be utilized immediately, or may be stored in local and/or remote memory for future use.

Once the patient cohort creation system has the plurality of patient records, and the lists of predetermined plurality of features for the different clinical diagnoses, conditions, or diseases from the clinical feature database, the patient cohort creation system can generate the database of extracted features for each of the plurality of different conditions, illnesses, diagnoses, and treatments. This database of extracted features can then be utilized by researchers and clinicians.

Accordingly, at step 130 of the method, the patient cohort creation system uses the identified plurality of features for the clinical diagnosis, condition, or disease to extract feature values from the plurality of patient records for the plurality of patients, for a first time period. The feature values may be extracted using any known mechanism, process, or program to identify and extract a feature value for a feature from a medical record. The first time period may be any time period, and may be a predetermined, programmed, default, learned, or any other time period. According to one embodiment, the time period is the full extent of time found within a plurality of records for a patient. For example, patient Pi may have records in the database for a period of 6 months, and therefore the system will extract feature values for the duration of that 6 months, associating a timestamp with extracted features. Similarly, patent P2 may have records in the database for a period of 4 months, and therefore the system will extract feature values for the duration of that 4 months, associating a timestamp with extracted features.

According to one embodiment, the time period is a predetermined time period of 3 months, and thus the patient cohort creation system extracts feature values from records for a time period of 3 months (beginning, according to just one possible example, with admission to a healthcare facility or diagnosis of a condition, among many, many other starting time points or events), associating a timestamp with extracted feature values.

Once patient cohort creation system 200 extracts feature values from the plurality of patient records, the extracted feature values can be utilized immediately, or may be stored in local and/or remote memory for future use. Accordingly, at step 140 of the method, the patient cohort creation system generates and/or populates a data structure such as a feature value table with the extracted feature values. The data structure can be any data structure sufficient to both store the extracted feature values and enable identification and retrieval of features and/or feature values from the data structure. The data structure can be stored in memory of the patient cohort creation system. The data structure, database, or memory storing the extracted feature values can be a local or remote database and can be in direct and/or indirect communication with the patient cohort creation system 200.

At step 150 of the method, the patient cohort creation system receives a user request for a patient cohort from the plurality of patients, wherein the patient cohort will be a subset of the plurality of patients, such as fewer than all of the plurality of patients. The user request can be received via a user interface of the patient cohort creation system. For example, a user request can be provided using any method for providing information to a computer system. The request may be provided by dictation, typing, selecting, clicking, or any other method of using a user interface. The user interface used to provide the user request may be a component of the patient cohort creation system, or may be remote to or a separate component from the patient cohort creation system. For example, the patient cohort creation system may be in wired and/or wireless communication to another device that comprises the user interface.

According to an embodiment, the user request comprises information that the patient cohort creation system will utilize to identify which patients from the plurality of patients should comprise the patient cohort. For example, the user request may comprise an identification of one of the plurality of clinical diagnoses, conditions, or diseases for which a list of features was generated. For example, the user request may also comprise an identification of a time period for the patient data and for the patient cohort. Thus, the user request may also comprise a predetermined time period that is some or all of the first time period discussed above.

According to an embodiment, the patient cohort creation system uses the received user request to identify a first patient cohort from the feature value table generated in step 140, for a predetermined time period, based on one of the plurality of clinical diagnoses, conditions, or diseases. The predetermined time period is some or all of the first time period, and the first patient cohort is a subset of the plurality of patients in the feature value table. The user request comprises the information necessary to identify the subset of the plurality of patients in the feature value table, as well as the predetermined time period.

Once patient cohort creation system identifies the first patient cohort from the feature value table, the identified first patient cohort can be utilized immediately, or may be stored in local and/or remote memory for future use.

At step 160 of the method, the patient cohort creation system provides the identified first patient cohort to the user via a user interface. The patient cohort creation system and user interface can provide the first patient cohort using any method for providing or reporting information. For example, the first patient cohort may be provided via a user interface of the patient cohort creation system, or may be communicated by wired and/or wireless communication to another device. The system may communicate the information to a mobile phone, computer, laptop, wearable device, and/or any other device configured to allow display and/or other communication of the information. The reported information may be a list of patients in the cohort along with the associated health records for each of the patients in the cohort, for the first time period.

At step 170 of the method, the patient cohort creation system receives a modification request from the user to modify the first patient cohort. The modification request can be received via a user interface of the patient cohort creation system. For example, a modification request can be provided using any method for providing information to a computer system. The request may be provided by dictation, typing, selecting, clicking, or any other method of using a user interface. The user interface used to provide the modification request may be a component of the patient cohort creation system, or may be remote to or a separate component from the patient cohort creation system. For example, the patient cohort creation system may be in wired and/or wireless communication to another device that comprises the user interface.

According to an embodiment, the modification request comprises information that the patient cohort creation system will utilize to identify how the first patient cohort should be modified. For example, the modification request may comprise a request to modify one or more of: (i) patients in the first patient cohort; (ii) the predetermined time period; and (iii) the predetermined plurality of features for the clinical diagnosis, condition, or disease. For example, the modification request may comprise a feature that broadens the first patient cohort and therefore allows more patients to be added to the first patient cohort. Alternatively, the modification request may comprise a feature that narrows the first patient cohort and therefore removes patients from the first patient cohort. For example, the modification request may comprise an identification of a new feature to add to the plurality of features for the clinical diagnosis, condition, or disease, and therefore may broaden or narrow the first patient cohort, depending on the identified new feature. As one example, requesting that the first patient cohort comprise new feature Lxi for condition X may result in more patients being added to the first patient cohort, or patients being removed from the first patient cohort. As another example, the modification request may comprise a request to shorten or lengthen the predetermined time period.

At step 180 of the method, the patient cohort creation system uses the received modification request to modify the first patient cohort to generate a modified patient cohort, which may comprise fewer or more patients. Notably, the patient cohort creation system modifies the first patient cohort without additional identification or extraction of clinical features from the plurality of patient records for the plurality of patients from the database. This is because the patient cohort creation system uses the information (i.e., the list of features and the extracted feature values) already populated in the feature values table to identify which patients to add or subtract from the first patient cohort to generate the modified patient cohort, based on the received modification request. This is one of the major advantages of this system over prior art systems. Generating and then utilizing a well-defined and well-populated feature values table allows for significantly faster analysis and generation of patient cohorts, improved analysis of patient cohorts, and so on. Since identification and extraction of feature values is one of the limiting steps during the creation of a patient cohort, having that information readily available for generation of a first patient cohort, or modification to generate a modified patient cohort, can essentially skip this step, vastly speeding up the system and resulting in improved processing efficiency when generating either the first patient cohort or the modified patient cohort.

Once patient cohort creation system generates the modified patient cohort from the feature value table, the modified patient cohort can be utilized immediately, or may be stored in local and/or remote memory for future use.

At step 190 of the method, the patient cohort creation system provides the modified patient cohort to the user via a user interface. The patient cohort creation system and user interface can provide the modified patient cohort using any method for providing or reporting information. For example, the modified patient cohort may be provided via a user interface of the patient cohort creation system, or may be communicated by wired and/or wireless communication to another device. The system may communicate the information to a mobile phone, computer, laptop, wearable device, and/or any other device configured to allow display and/or other communication of the information. The reported information may be a list of patients in the cohort along with the associated health records for each of the patients in the cohort, for the first time period or a modified time period.

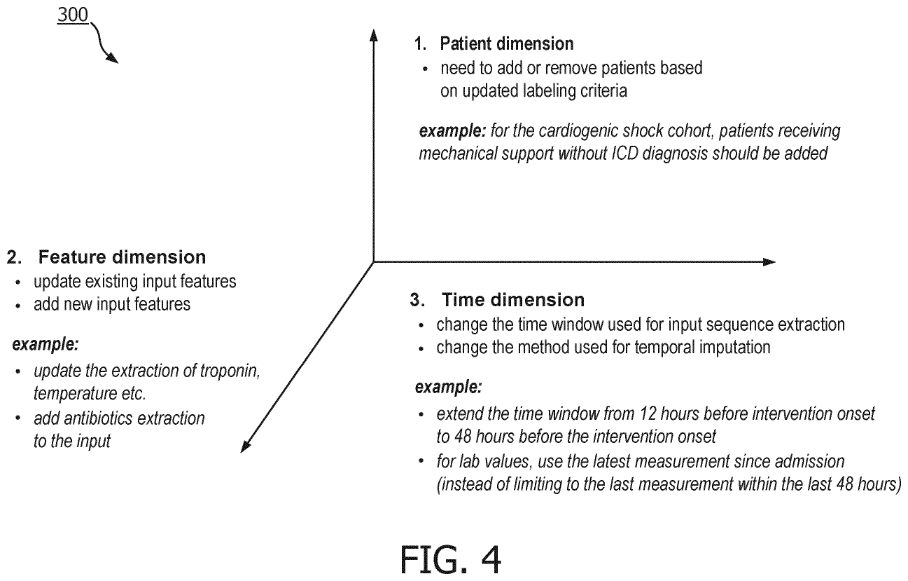

Referring to FIG. 4, in one embodiment, is a graphic 300 illustrating complexities induced by modifications of a patient cohort in all three dimensions, including the patient dimension, the feature dimension, and the time dimension.

According to one example, a condition is sepsis such as prediction or determination of sepsis. The patient dimension may comprise sepsis and/or non-sepsis patients; the feature dimension may comprise WBC counts, lactate, and/or other features used to predict or determine sepsis; the time dimension may comprise six hours before the onset of sepsis. Each one of these dimensions can be determined by the user via the user request or modification, and any of these dimensions can be changed resulting in a different or modified patient cohort. As just one example of a modification, the time dimension can be changed by extending or shortening the time window before sepsis, such as to 4 or 8 hours.

The patient cohort creation system automates the data extraction process so that investigators can spend minimal efforts on data extraction. For example, the system avoids the inefficiency of re-running the data extraction process whenever there is any modification of the problem definition. According to one possible embodiment, the patient cohort creation system comprises a data extraction pipeline containing multiple APIs of create/add/remove/update the patient/feature/time dimension; and a public shared repository storing implementations of labelling different clinical conditions.

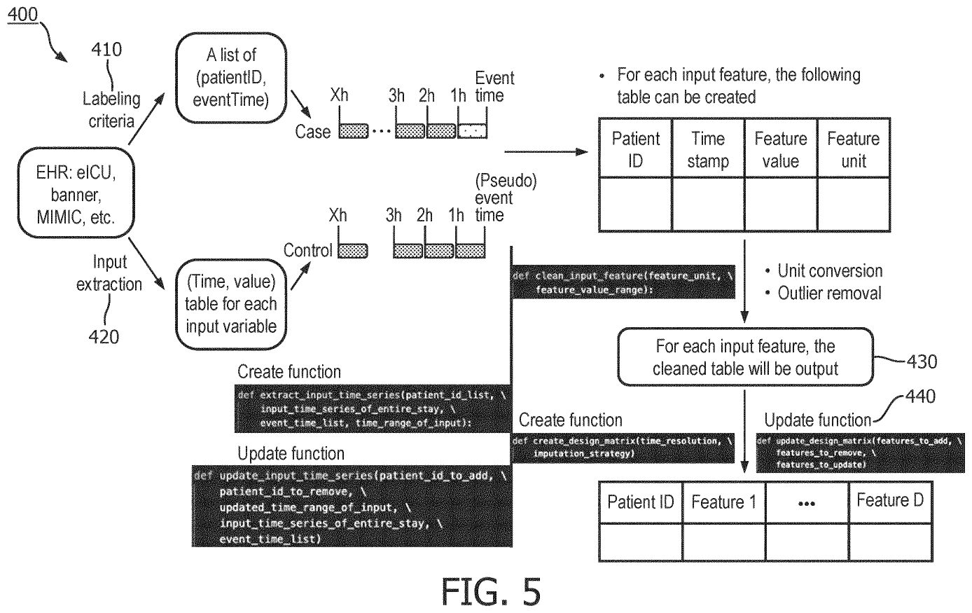

Referring to FIG. 5, in one embodiment, is a graphic 400 illustrating a data extraction pipeline of a patient cohort creation system. This data extraction pipeline is just one possible embodiment, and is provided as a non-limiting example.

At step 410 of the method, labeling criteria are applied. First, starting from an EHR data source, such as MIMIC-III or eICU, a list of (patientID, eventTime) can be extracted using the labelling criteria. This includes both the patient cases (using the inclusion criteria) and controls (using the exclusion criteria). This step can be automated by creating a public shared repository containing the implementations of common clinical conditions, which is described in greater detail elsewhere herein.

At step 420 of the method, a time series is created or updated for input variables of the selected patient set. Given a list of input variables, the (timeStamp, value) table for each input variable can also be extracted. This creation step can be automated by the following API:

def extract_input_time series(patient_id_list, input_time_series_of_entire_stay, event time_list, time_range_of_input) where patient_id_list is the set of case/control patients identified from the first step, input_time_series_of_entire_stay is the entire time series of multiple clinical variables available in the HER; event_time_list is the list of onset time of the clinical event, such as onset time of sepsis, identified from the first step; and time_range_of_input is the time range of interest used to build the predictive models, such as 12 to 1 hour before the onset time of the clinical event.

According to an embodiment, the output of this API will be a time series for each feature of each patient. The user can also update the existing time series using the following API:
def update_input_time series(patient_id_to_add, patient_id_to_remove, updated_time_range_of_input, input_time_series_of_entire stay, event_time_list)
   where patient_id_to_add is the list of patient ID to add in the updated cohort; patient_id_to_remove is the list of patient ID to remove in the updated cohort; updated_time_range_of_input is the updated time range in the updated cohort; input_time_series_of_entire_stay is the entire time series of multiple clinical variables available in the HER; and event_time_list is the list of onset time of the clinical event, such as onset time of sepsis, identified from the first step.

According to an embodiment, the output of step 420 will be a table for each input feature with the following columns: patient ID, time stamp when the feature gets measured, the measured feature value, and the unit of the feature value.

At step 430 of the method, input features are cleaned. For example, given the output table for each feature from step 420, the following API can be used to clean each input feature by applying feature-specific unit conversion and outlier removal:
def clean_input_feature(feature_unit, feature_value_range)
   where feature unit is the available feature unit of these measured features, and feature_value_range is the plausible range of these input features.

For each input feature, there are a limited set of input features and their relationships are available in the literature. These can be easily implemented in a look-up table. Similarly, the plausible range of typical vitals and labs can also be implemented in a look-up table. Furthermore, given each feature name or unit can be documented in different ways, these documentation patterns can be extracted once for each data source and then save them in a look-up table. In summary, this step can also be automated. According to an embodiment, the output of step 430 is a cleaned table for each input feature.

At step 440 of the method, a patient design matrix is created or updated. The following API can be used to create the patient design matrix from multiple tables from multiple input features:
def create_design_matrix(time_resolution, imputation_strategy)
   where time_resolution specifies which time points will be used to create the patient design matrix; and imputation_strategy is the imputation strategy used to deal with missing values in the design matrix.

After the creation of the design matrix, it can also be updated using the following API: def update design matrix(features_to_add, features to remove, features_to_update) were features_to_add is the new features to be added to the patient design matrix; features to remove is the features to be removed from the patient design matrix; and features_to_update is the features that need to be updated from the patient design matrix.

According to an embodiment, the output of step 440 is a final patient design matrix that can be used for model training.

According to an embodiment, step 410 of the method can potentially be the most difficult or challenging step due to the varieties of labelling criteria for multiple clinical conditions. However, given that there often exist consensus definitions for a given disease, they can be implemented in the public repository and reused by different investigators. Examples include Sepsis-III criteria for sepsis, and Berlin definition for acute respiratory distress syndrome (ARDS).

For each contribution to the repository, a wiki page can be used to document: (1) the labelling criteria being implemented; (2) the data source used to test the labelling criteria: and (3) summary statistics from applying the labelling criteria to the data source. As for the programming language, both Python and SQL can be used due to their wide acceptance in the data science community, among other languages.

According to an embodiment, therefore, the patient cohort creation system can be applied by data science investigators to extract patient cohorts from electronic health records. Due to its nature, it can be widely used in building any disease prediction model, such as hemodynamic instability, sepsis, infection, respiratory distress, and many others. The patient cohort creation method can be applied, for example, in the algorithm development phase. The developed algorithm can be deployed to patient monitors, for example. Further, the patient cohort creation system can save investigator time on data extraction, which often takes enormous amounts of time and efforts, and spend more efforts on algorithm development and validation. It will also facilitate the delivery of disease risk prediction models of higher quality.

Referring to FIG. 3 is a schematic representation of a patient cohort creation system. System 200 may be any of the systems described or otherwise envisioned herein, and may comprise any of the components described or otherwise envisioned herein. It will be understood that FIG. 3 constitutes, in some respects, an abstraction and that the actual organization of the components of the system 200 may be different and more complex than illustrated.

According to an embodiment, system 200 comprises a processor 220 capable of executing instructions stored in memory 230 or storage 260 or otherwise processing data to, for example, perform one or more steps of the method. Processor 220 may be formed of one or multiple modules. Processor 220 may take any suitable form, including but not limited to a microprocessor, microcontroller, multiple microcontrollers, circuitry, field programmable gate array (FPGA), application-specific integrated circuit (ASIC), a single processor, or plural processors.

Memory 230 can take any suitable form, including a non-volatile memory and/or RAM. The memory 230 may include various memories such as, for example L1, L2, or L3 cache or system memory. As such, the memory 230 may include static random access memory (SRAM), dynamic RAM (DRAM), flash memory, read only memory (ROM), or other similar memory devices. The memory can store, among other things, an operating system. The RAM is used by the processor for the temporary storage of data. According to an embodiment, an operating system may contain code which, when executed by the processor, controls operation of one or more components of system 200. It will be apparent that, in embodiments where the processor implements one or more of the functions described herein in hardware, the software described as corresponding to such functionality in other embodiments may be omitted.

User interface 240 may include one or more devices for enabling communication with a user. The user interface can be any device or system that allows information to be conveyed and/or received, and may include a display, a mouse, and/or a keyboard for receiving user commands. In some embodiments, user interface 240 may include a command line interface or graphical user interface that may be presented to a remote terminal via communication interface 250. The user interface may be located with one or more other components of the system, or may located remote from the system and in communication via a wired and/or wireless communications network.

Communication interface 250 may include one or more devices for enabling communication with other hardware devices. For example, communication interface 250 may include a network interface card (NIC) configured to communicate according to the Ethernet protocol. Additionally, communication interface 250 may implement a TCP/IP stack for communication according to the TCP/IP protocols. Various alternative or additional hardware or configurations for communication interface 250 will be apparent.

Storage 260 may include one or more machine-readable storage media such as read-only memory (ROM), random-access memory (RAM), magnetic disk storage media, optical storage media, flash-memory devices, or similar storage media. In various embodiments, storage 260 may store instructions for execution by processor 220 or data upon which processor 220 may operate. For example, storage 260 may store an operating system 261 for controlling various operations of system 200.

It will be apparent that various information described as stored in storage 260 may be additionally or alternatively stored in memory 230. In this respect, memory 230 may also be considered to constitute a storage device and storage 260 may be considered a memory. Various other arrangements will be apparent. Further, memory 230 and storage 260 may both be considered to be non-transitory machine-readable media. As used herein, the term non-transitory will be understood to exclude transitory signals but to include all forms of storage, including both volatile and non-volatile memories.

While system 200 is shown as including one of each described component, the various components may be duplicated in various embodiments. For example, processor 220 may include multiple microprocessors that are configured to independently execute the methods described herein or are configured to perform steps or subroutines of the methods described herein such that the multiple processors cooperate to achieve the functionality described herein. Further, where one or more components of system 200 is implemented in a cloud computing system, the various hardware components may belong to separate physical systems. For example, processor 220 may include a first processor in a first server and a second processor in a second server. Many other variations and configurations are possible.

According to an embodiment, the electronic medical record system 270 is an electronic medical records database from which the information about a plurality of patients, including demographic, diagnosis, and/or treatment information may be obtained or received. According to an embodiment, the electronic medical record system 270 is an electronic medical records database from which training data can be obtained or received. The training data can be any data that will be utilized to create a patient cohort to train an algorithm. The training data can comprise any other information. The electronic medical records database may be a local or remote database and is in direct and/or indirect communication with system 200. Thus, according to an embodiment, the clinical risk prediction system comprises an electronic medical record database or system 270.

According to an embodiment, storage 260 of system 200 may store one or more algorithms, modules, and/or instructions to carry out one or more functions or steps of the methods described or otherwise envisioned herein. For example, the system may comprise, among other instructions or data, patient data 262, feature lists 263, feature extraction instructions 264, a feature value table 265, and/or reporting instructions 266.

According to an embodiment, the patient data 262 is plurality of patient records for a plurality of patients and is obtained from a database, such as database 270. The patient records can comprise any data about a patient. For example, the patient records may comprise demographic information, diagnostic information, clinical measurements, treatment information, and/or any other information, including over a period of time for each of the plurality of patients. According to an embodiment, the patient records comprises clinical data that measures patient physiology such as physiological measurements of vital signs, lab results, imaging results, and other data and event logs-such as timestamps of orders of labs, imaging, medication, consultation, and other data-that can reflect the procedures the patient received in a care setting. The patient records can comprise any other information.

According to an embodiment, the feature lists 263 are list of features for each of a plurality of different clinical diagnoses, conditions, or diseases. The feature lists for the plurality of different clinical diagnoses, conditions, or diseases may be stored in and/or received from one or more databases. For example, the clinical features database comprises a list of features L_{X} relevant to condition X, where the list of features L_{X} comprises a plurality of features that are known to be relevant to, or predicted to be relevant to, or otherwise associated with, condition X. List of features L_{X} may be, for example, ten different features for condition X (age, sex, antibiotic regimen, etc.). Similarly, the clinical features database comprises a list of features L_{z} relevant to condition Z, where the list of features L_{z} comprises a plurality of features that are known to be relevant to, or predicted to be relevant to, or otherwise associated with, condition Z. And so on for a wide variety of different diagnoses, conditions, illnesses, or diseases, and so on.

According to an embodiment, the feature extraction instructions 264 direct the system to extract feature values from the plurality of patient records for the plurality of patients, for a first time period, using an identified plurality of features for a clinical diagnosis, condition, or disease. The feature values may be extracted using any known mechanism, process, or program to identify and extract a feature value for a feature from a medical record. The first time period may be any time period, and may be a predetermined, programmed, default, learned, or any other time period. According to one embodiment, the time period is the full extent of time found within a plurality of records for a patient.

According to an embodiment, the feature value table 265 is a data structure with the extracted feature values. The data structure can be any data structure sufficient to both store the extracted feature values and enable identification and retrieval of features and/or feature values from the data structure. The data structure can be stored in memory of the patient cohort creation system.

According to an embodiment, the reporting instructions 266 direct the system to generate and provide to a user via a user interface information comprising a first patient cohort and/or a modified patient cohort. Alternatively, the information may be communicated by wired and/or wireless communication to another device. For example, the system may communicate the information to a mobile phone, computer, laptop, wearable device, and/or any other device configured to allow display and/or other communication of the information.

According to an embodiment, the patient cohort creation system is configured to process many thousands or millions of datapoints in the patient data to identify and extract lists of features for a plurality of different clinical diagnoses, conditions, or diseases, and to populate the features value table, and to generate a patient cohort, and to modify the patient cohort. For example, generating a functional patient cohort according to the claimed method and system requires processing of millions of datapoints from input data. This can require millions or billions of calculations to generate a patient cohort. Thus, generating a patient cohort comprises a process with a volume of calculation and analysis that a human brain cannot accomplish in a lifetime, or multiple lifetimes.

By providing an improved patient cohort creation system as described or otherwise envisioned herein, the novel patient cohort creation system has an enormous positive effect on processing efficiency, researcher time, basic research itself, and on patient care compared to prior art systems.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

1. A method for generating a modified patient cohort from a patient records database, the method comprising:
receiving, from the patient records database, the plurality of patient records for the plurality of patients;
retrieving, from a clinical feature database, a predetermined plurality of features for a clinical diagnosis, condition, or disease, wherein the clinical feature database comprises a predetermined plurality of features for each of a plurality of clinical diagnoses, conditions, or diseases;
extracting, using the predetermined plurality of features for the clinical diagnosis, condition, or disease, clinical features from at least one of the plurality of patient records for the plurality of patients to generate a clinical feature dataset for a first time period;
generating a feature value table in memory comprising the extracted clinical feature dataset;
identifying, in response to a user request for a patient cohort based on one of the plurality of clinical diagnoses, conditions, or diseases, a first patient cohort from the feature value table for a predetermined time period, wherein the predetermined time period is some or all of the first time period, and wherein the first patient cohort is a subset of the plurality of patients in the feature value table;
providing, via a user interface, the first patient cohort to the user;
receiving a modification request from the user to modify the first patient cohort, wherein the modification comprises a modification of one or more of: (i) patients in the first patient cohort;
(ii) the predetermined time period; and (iii) the predetermined plurality of features for the clinical diagnosis, condition, or disease;
modifying, based on the received modification request and without additional extraction of clinical features, the first patient cohort to generate the modified patient cohort; and
providing, via the user interface, the modified patient cohort to the user.

2. The method of claim 1, wherein the feature value table comprises, for each of a plurality of patients, a patient identification and a feature value for some or all of the predetermined plurality of features for the clinical diagnosis, condition, or disease.

3. The method of claim 1, wherein the first patient cohort and the modified patient cohort comprises a list of patients each identified by a patient ID, and, for each patient, a feature value for each of the predetermined plurality of features.

4. The method of claim 1, wherein the modified patient cohort comprises more patients compared to the first patient cohort.

5. The method of claim 1, wherein the modified patient cohort comprises one or more additional features compared to the first patient cohort.

6. The method of claim 1, wherein the modified patient cohort comprises a longer or shorter time period compared to the first patient cohort.

7. The method of claim 1, wherein the modification request received from the user comprises an identification of a feature not originally listed in the predetermined plurality of features for the clinical diagnosis, condition, or disease.

8. The method of claim 1, wherein the predetermined time period is a time period before onset of a clinical condition.

9. A system for generating a modified patient cohort, the system comprising:
a patient records_database comprising a plurality of patient records for a plurality of patients;
a clinical feature database comprising a predetermined plurality of features for each of a plurality of clinical diagnoses, conditions, or diseases;
a user interface; and
a processor configured to:
(i) retrieve, from a clinical feature database, a predetermined plurality of features for a clinical diagnosis, condition, or disease;
(ii) extract, using the predetermined plurality of features for the clinical diagnosis, condition, or disease, clinical features from at least one of the plurality of patient records for the plurality of patients to generate a clinical feature dataset for a first time period;
(iii) generate a feature value table in memory comprising the extracted clinical feature dataset;
(iv) identify, in response to a user request for a patient cohort based on one of the plurality of clinical diagnoses, conditions, or diseases, a first patient cohort from the feature value table for a predetermined time period, wherein the predetermined time period is some or all of the first time period, and wherein the first patient cohort is a subset of the plurality of patients in the feature value table;
(v) provide, via the user interface, the first patient cohort to the user;
(vi) receive a modification request from the user to modify the first patient cohort, wherein the modification comprises a modification of one or more of: patients in the first patient cohort; the predetermined time period; and the predetermined plurality of features for the clinical diagnosis, condition, or disease;
(vii) modify, based on the received modification request and without additional extraction of clinical features, the first patient cohort to generate a modified patient cohort; and
(viii) provide, via the user interface, the modified patient cohort to the user.

10. The system of claim 9, wherein the feature value table comprises, for each of a plurality of patients, a patient identification and a feature value for some or all of the predetermined plurality of features for the clinical diagnosis, condition, or disease.

11. The system of claim 9, wherein the first patient cohort and the modified patient cohort comprises a list of patients each identified by a patient ID, and, for each patient, a feature value for each of the predetermined plurality of features.

12. The system of claim 9, wherein the modified patient cohort comprises more patients compared to the first patient cohort.

13. The system of claim 9, wherein the modified patient cohort comprises one or more additional features compared to the first patient cohort.

14. The system of claim 9, wherein the modified patient cohort comprises a longer or shorter time period compared to the first patient cohort.

15. The system of claim 9, wherein the modification request received from the user comprises an identification of a feature not originally listed in the predetermined plurality of features for the clinical diagnosis, condition, or disease.

16. A non-transitory computer-readable storage medium having stored a computer program comprising instructions, which, when executed by a processor, cause the processor to:
receive, from the patient records database, the plurality of patient records for the plurality of patients;
retrieve, from a clinical feature database, a predetermined plurality of features for a clinical diagnosis, condition, or disease, wherein the clinical feature database comprises a predetermined plurality of features for each of a plurality of clinical diagnoses, conditions, or diseases;
extract, using the predetermined plurality of features for the clinical diagnosis, condition, or disease, clinical features from at least one of the plurality of patient records for the plurality of patients to generate a clinical feature dataset for a first time period;
generate a feature value table in memory comprising the extracted clinical feature dataset;
identify, in response to a user request for a patient cohort based on one of the plurality of clinical diagnoses, conditions, or diseases, a first patient cohort from the feature value table for a predetermined time period, wherein the predetermined time period is some or all of the first time period, and wherein the first patient cohort is a subset of the plurality of patients in the feature value table;
provide, via a user interface, the first patient cohort to the user;
receive a modification request from the user to modify the first patient cohort, wherein the modification comprises a modification of one or more of: (i) patients in the first patient cohort; (ii) the predetermined time period; and (iii) the predetermined plurality of features for the clinical diagnosis, condition, or disease;
modify, based on the received modification request and without additional extraction of clinical features, the first patient cohort to generate a modified patient cohort; and
provide, via the user interface, the modified patient cohort to the user.

17. The non-transitory computer-readable storage medium of claim 16, wherein the feature value table comprises, for each of a plurality of patients, a patient identification and a feature value for some or all of the predetermined plurality of features for the clinical diagnosis, condition, or disease.

18. The non-transitory computer-readable storage medium of claim 16, wherein the first patient cohort and the modified patient cohort comprises a list of patients each identified by a patient ID, and, for each patient, a feature value for each of the predetermined plurality of features.

19. The non-transitory computer-readable storage medium of claim 16, wherein the modified patient cohort comprises at least one of:
more patients compared to the first patient cohort,
one or more additional features compared to the first patient cohort, or
a longer or shorter time period compared to the first patient cohort.

20. The non-transitory computer-readable storage medium of claim 16, wherein the modification request received from the user comprises an identification of a feature not originally listed in the predetermined plurality of features for the clinical diagnosis, condition, or disease.
